(19) United States
(12) Patent Application Publication
(10) Pub. No.: US 2016/0213604 A1
(43) Pub. Date: Jul. 28, 2016

(54) COMPOSITIONS AND METHODS FOR REDUCING CARIES-CAUSING ORAL BACTERIA

(71) Applicant: Apollonia Health Inc., Hamilton (CA)

(72) Inventors: Joon Kim, Hamilton (CA); Dennis Gerard Cvitkovitch, Oakville (CA)

(21) Appl. No.: 14/668,142

(22) Filed: Mar. 25, 2015

Related U.S. Application Data
(63) Continuation of application No. PCT/CA2015/000038, filed on Jan. 23, 2015.

(51) Int. Cl. A61K 8/97; A61Q 11/00; A23G 3/48; A61K 8/64

(52) U.S. Cl. CPC A61K 8/97; A61K 8/64; A61Q 11/00; A23G 3/48; A23V 2002/00

(57) ABSTRACT

Provided are compositions comprising an orally acceptable carrier, an extract of Myristica fragrans derived from nutmeg or mace, and an extract of Vaccinium subgenus Oxycoccus. The compositions may also include an extract of neem and Competence Stimulating Peptide (CSP). Also provided is a method for inhibiting the growth of Streptococcus mutans in a biofilm in the oral cavity of a subject, the method comprising administering to the oral cavity of the subject a composition comprising an orally acceptable carrier, an extract of Myristica fragrans derived from nutmeg or mace, an extract of Vaccinium subgenus Oxycoccus, wherein administering the composition selectively inhibits the rate of growth of Streptococcus mutans over the rate of growth of at least one species of non-Streptococcus mutans bacteria in the biofilm.

### CROSS-REFERENCE

This application is a continuation application of PCT International Patent Application No. PCT/CA2015/000038 filed Jan. 23, 2015 which claims the benefit of U.S. Provisional Application Nos. 61/930,685 and 61/930,725 filed Jan. 23, 2014, the contents of which are incorporated by reference.

### INCORPORATION-BY-REFERENCE OF MATERIAL ELECTRONICALLY SUBMITTED

Incorporated by reference in its entirety is a computer-readable nucleotide/amino acid sequence listing submitted concurrently herewith and identified as follows: 626 bytes ASCII (Text) file named “SequenceListing_ST25.txt” created Mar. 24, 2015.

### FIELD OF THE INVENTION

This invention relates to compositions and methods for treating and/or inhibiting the formation of dental caries. In particular, the invention relates to compositions comprising herbal extracts for treating and/or inhibiting the formation of dental caries.

### BACKGROUND

S. mutans is the principal etiological agent of dental caries in humans. Dental caries is an undesirable condition of the oral cavity and, over the years, has remained an intractable disease. Dental caries is a unique multifactorial infectious disease. Dental caries affects teeth at all levels and can cause extensive crown mutilations, bacterial disorders of the periapical tissues, or even loss of the affected dental elements. Clinically, the disease is characterized by demineralization of the dental enamel and of the dentin in various stages of progress until it affects the pulp space. When the lesion passes beyond the enamel-dentin border, a phlogistic reaction of the pulp tissues is constantly observed, with the formation of reaction dentin in some cases.

Recently it has become clear that dental plaque actually consists of hundreds of different bacterial taxa. Most of these bacteria exist on the surface of teeth in heterogeneous communities called plaque or biofilms. The mouth thus acts as a reservoir for these bacteria. While most of these bacteria are commensal, meaning they fail to adversely affect the human host, others are pathogenic and can cause tooth decay. Moreover these pathogenic bacteria have been found to cause a life-threatening disease called endocarditis.

As noted above, among all oral bacteria, the primary etiologic agent of tooth decay is the bacterium S mutans. S. mutans is a resident of the biofilm environment of dental plaque, a matrix of bacteria and extracellular material that adheres to the tooth surface. Under appropriate environmental conditions, populations of S mutans will rise and the pH of the surrounding plaque will drop. S. mutans, being among the most acid tolerant organisms residing in dental plaque, will increase in numbers in this acidic environment and eventually become a dominant member of the plaque community. Once attached, S. mutans inflicts its damage by fermenting simple dietary sugars into lactic acid. This situation eventually leads to dissolution of the tooth enamel, resulting in the development of dental caries. Since there is a strong correlation between the proportion of S. mutans in dental plaque or in saliva relative to other bacterial species and the presence or risk of future outbreaks of dental caries, S. mutans in plaque or saliva may serve as an index for both dental caries activity state and dental caries risk or susceptibility. These indices play an increasingly important role in the diagnosis and treatment of dental caries.

Most oral bacteria are benign and are actually important for oral health, such as Streptococcus oralis and Lactobacillus casei. Consequently, an overall reduction in oral bacteria is not a practical means to prevent tooth decay. For example, when the level of certain oral bacteria is reduced (e.g., after prolonged use of antibiotics), an overgrowth of indigenous yeast occurs, resulting in a disorder known as Thrush.

Approximately 50% of adults have at least four caries-related lesions that have been treated or require treatment, and approximately 30% of adults have over 50% of their teeth affected by dental caries. Previous efforts toward the correction of dental caries have revolved around the use of the standard toothbrush to remove dental plaque. Also in widespread use today are electric brushes, floss and adjuncts such as proxy brushes. In addition, numerous toothpastes and mouth rinses containing various supplements are touted as aids in the prevention of dental caries. For example, fluoride is commonly sold as a product for slowing the process of dental decay. However, the efficacy of such methods of treating or preventing dental caries is questionable. Dental plaque can only partially be removed from the oral cavity, even when a demanding regimen of oral hygiene that may include flossing, brushing and regular visits to a dentist is followed. In addition, many toothpastes and mouth rinses contain toxic supplements, such as fluoride and triclosan, which can be toxic to very young children.

S. mutans has also been found to contribute to infective endocarditis. Infective endocarditis is a potentially lethal infection of both native (normal) and artificial heart valves, and if left untreated can be fatal. S. mutans forms biofilms on the surface of these valves, and are typically a mixed community of a variety of pathogenic bacteria. Since these bacteria find their way to the valves via the blood stream and the blood stream is typically aseptic, there are usually few opportunities for bacteria to cause these infections. One exception is when pathogenic bacteria, like S. mutans, enter the blood stream during dental procedures. Hence, the probability of infection is directly related to the reservoir of infectious bacteria found in the oral cavity.

Since S. mutans is recalcitrant to antibiotic therapy in the biofilm phase, preventive approaches are considered safer and more practical. Currently, these approaches rely mainly on prophylactic antibiotic therapy in high-risk patients. However, such treatments merely control the spreading of free bacteria (planktonic) and acute infections. Hence, the infections tend to be persistent, requiring multiple rounds of treatments to avoid permanent and lethal valve damage.

U.S. Patent Application Publication No. 2002/0081302, published Jun. 27, 2002 to Cvitkovitch, Dennis et al. for “Signal Peptides, Nucleic Acid Molecules and Methods For Treatment of Caries” provides a compound that competitively inhibits binding of CSP to S. mutans histidine kinase. The disclosed compound is generally a peptide or an antibody, and preferably a derivative of a particular specified SEQ ID, a fragment of that SEQ ID or a derivative of a fragment of that SEQ ID.

U.S. Patent Application Publication No 2004/0105824 published Jun. 3, 2004 to Goodman, Steven D. et al for “Preventing Tooth Decay and Infective Endocarditis Using Natural Oligopeptides” provides compositions, medicaments, and methods for the treatment or prophylaxis of conditions associated with the binding of S. mutans to teeth. Specifically, the publication teaches a method for preventing dental caries and infective endocarditis in a subject by treating the subject's oral cavity with a composition or medicament comprising the competence stimulating peptide (CSP) of the causative bacterium, which inhibits the ability of the said bacteria to attach to the surface of teeth. The anti-caries compositions comprised CSP and sucrose.

U.S. Patent Publication No. 2005/152853 for “Signal Peptides, Nucleic Acid Molecules and Methods For Treatment of Caries”, to Huang Yi-Chen et al. and The Governing Council of the University of Toronto discloses compounds that competitively inhibit binding of CSP to S. mutans histidine kinase. The compounds are generally a peptide or an antibody, and are preferably a derivative of specified SEQ IDs.

U.S. Pat. No. 6,024,958, issued Feb. 15, 2000 to Lehner et al for “Defined Peptide Subunits of Streptococcus Mutans Antigen I/II” discloses agents useful to prevent and treat dental caries either by eliciting an immunological response or by preventing adhesion of S. mutans to the tooth. The agents are polypeptides or extended polypeptides extended at the N-terminus or C-terminus or both with a non-wild-type amino acid sequence, the polypeptide being selected from one of 11 specified SEQ IDs. Such polypeptides or extended polypeptides are in the N-terminal acylated and/or C-terminal amidated form. The patent also teaches a pharmaceutical composition comprising that polypeptide or that extended polypeptide, as well as an immunological composition comprising that polypeptide or that extended polypeptide along with an immunologically acceptable carrier.

W02007/053945 for “Oral Anti-Microbial Compositions Comprising Competence-Stimulating Peptide” by Kane Biotech Inc. provides compositions and methods for inhibiting the growth and formation of biofilms. The compositions and methods include a combination of at least one antimicrobial compound and at least one CSP analogue or CSP.

W02011/068813 for “Oral Compositions Containing Extracts of Myristica fragrans and Related Methods” by Colgate-Palmolive Company provides compositions formed by the addition of extract of Myristica fragrans to various dentifrice compositions resulting in tooth paste, mouth rinses, gums, mouth strips, beads, and other compositions that were suitable for treating and preventing a variety of oral diseases including gingivitis, plaque build-up, and the like. The extract of Myristica fragrans is thought to contain varying amounts, inter alia, of one or more of camphenes, limonenes, α- and β-pinenes, eugenol, methyl eugenol, isoeugenol, butyl benzoate, myristin, elemicin, a-terpineol, I3-phellandrene, myristic acid, butyl dodecanoate, a-caryophyllene alcohol, geranylacetone, and mixtures thereof, and other beneficial chemicals. The extract can be added to dentifrice compositions so that the amount delivered to the oral cavity upon use is effective to provide an antibacterial, antioxidant, and/or anti-inflammatory effect, as well as being effective to treat dry mouth (xerostomia). The components of extract of Myristica fragrans are taught to be combined with natural extracts other than Myristica fragrans, to provide enhanced activity. It also taught that dentifrices formulated with the extract of Myristica fragrans, in combination with natural extracts other than Myristica fragrans, exhibit antibacterial, anti-inflammatory, and/or antioxidant properties, and have been found effective in treating xerostomia, without the need for an additional antibacterial agent. Thus, an oral composition is provided comprising an extract from Myristica fragrans, an orally acceptable carrier, and a natural extract other than the extract from Myristica fragrans namely extracts comprising a mixture of extracts from at least three of Punica granatum, Myristica fragrans, Zingiber officinale, and Zizyphus joazeiro, and an orally acceptable carrier.

WO 2012/2034090 for “Compositions and Methods For the Removal of Biofilms” is based on the discovery that polypeptides that have one or more HMG-box domain(s), such as HMGB1, can interfere with the structure of extracellular DNA scaffold inside biofilms. By competing with microbial proteins that bind to the DNA scaffold in the biofilm, these polypeptides destabilize the biofilm, leading to destruction and removal of the biofilm by the host immune system.

There is a need to lower the effective concentrations of constituents of herbal compositions for treating or inhibiting the formation of dental caries.

### SUMMARY

In a first aspect, provided is a composition comprising an orally acceptable carrier and: an extract of Myristica fragrans derived from nutmeg or mace; and an extract of Vaccinium subgenus Oxycoccus.

The extract of Myristica fragrans and the extract of Vaccinium can be present in an amount selected to treat dental caries or inhibit the formation of dental caries by inhibiting the growth of Streptococcus mutans. The extract of Myristica fragrans and the extract of Vaccinium can be present in an amount selected to inhibit the growth of Streptococcus mutans in an oral biofilm. The selected amount can selectively inhibit the rate of growth of Streptococcus mutans over the rate of growth of at least one species of non-Streptococcus mutans bacteria. The selected amount can induce proliferation of the at least one species of non-Streptococcus mutans bacteria or have no effect on the rate of growth of the at least one species of non-Streptococcus mutans bacteria. The selected amount can decrease the rate of growth of the at least one species of non-Streptococcus mutans bacteria. The Streptococcus mutans and the at least one species of non-Streptococcus mutans bacteria can be in a biofilm comprising a mixed bacterial population. The at least one species of non-Streptococcus mutans bacteria can be Streptococcus oralis.

The composition can further comprise Competence Stimulating Peptide (CSP). The CSP can be present in an amount selected to selectively reduce the attachment of Streptococcus mutans to teeth. The CSP can be present in an amount ranging from about 0.05% to about 50% w/w.

The composition can be formulated for topical application in the mouth. The composition can be formulated as a mouthwash solution. The composition can be formulated as a denture wash. The composition can be formulated as a denture adhesive or denture cement. The composition can be formulated as a chewing gum. The composition can be formulated as a candy. The composition can be formulated as a beverage. The composition can further comprise an extract of other members of the Vaccinium genus, for example bilberry, blueberry, or huckleberry. The composition can further comprise an extract of neem.

In a second aspect, provided is a composition comprising an orally acceptable carrier and: an extract of Myristica fragrans derived from nutmeg or mace; and an extract of neem. The composition can further comprise an extract of Vaccinium subgenus Oxycoccus.

In a third aspect, provided is a method for inhibiting the growth of Streptococcus mutans in a biofilm in the oral cavity of a subject, the method comprising administering to the oral cavity of the subject a composition comprising an orally acceptable carrier and: an extract of Myristica fragrans derived from nutmeg or mace; and an extract of Vaccinium subgenus Oxycoccus; wherein administering the composition selectively inhibits the rate of growth of Streptococcus mutans over the rate of growth of at least one species of non-Streptococcus mutans bacteria in the biofilm.

Administering the composition can induce proliferation of the at least one species of non-Streptococcus mutans bacteria or have no effect on the rate of growth of the at least one species of non-Streptococcus mutans bacteria.

In a fourth aspect, provided is use of a composition to inhibit the growth of Streptococcus mutans in a biofilm in the oral cavity of a subject, the composition comprising an orally acceptable carrier and: an extract of Myristica fragrans derived from nutmeg or mace; and an extract of Vaccinium subgenus Oxycoccus; wherein administering the composition selectively inhibits the rate of growth of Streptococcus mutans over the rate of growth of at least one species of non-Streptococcus mutans bacteria in the biofilm.

Administering the composition can induce proliferation of the at least one species of non-Streptococcus mutans bacteria or have no effect on the rate of growth of the at least one species of non-Streptococcus mutans bacteria.

### BRIEF DESCRIPTION OF THE DRAWINGS

Embodiments of the present invention will be described with reference to the accompanying drawings, wherein like reference numerals denote like parts, and in which:

FIG. 1 shows the results of a Minimum Biofilm Eliminating Concentration (MBEC) protocol treating S. mutans biofilms with serially diluted nutmeg extract in the presence or absence of 1 μg/ml Competence-Stimulating Peptide (“CSP”);

FIG. 2 shows the results of an MBEC protocol treating S. mutans biofilms with serially diluted cranberry extract in the presence or absence of 1 μg/ml CSP;

FIG. 3 shows the results of an MBEC protocol treating S. mutans biofilms with serially diluted neem extract in the presence or absence of 1 μg/ml CSP;

FIG. 4 shows the results of an MBEC protocol treating S. mutans biofilms with a combination of serially diluted cranberry and neem extracts;

FIG. 5 shows the results of an MBEC protocol treating S. mutans biofilms with a combination of 1 μg/ml CSP and serially diluted nutmeg and neem extracts;

FIG. 6 shows the results of an MBEC protocol treating S. mutans biofilms with serially diluted nutmeg extract extracted with 95% ethanol, 40% ethanol, acetone, or water, the treatment in the presence or absence of 1 μg/ml CSP;

FIG. 7 shows the results of an MBEC protocol treating S. mutans biofilms with serially diluted cranberry extract extracted with 95% ethanol, 70% ethanol, acetone, of water, the treatment in the presence or absence of 1 μg/ml CSP;

FIG. 8 shows the results of an MBEC protocol treating S. mutans biofilms with serially diluted neem extract extracted with 95% ethanol, 40% ethanol, acetone, or water, the treatment in the presence or absence of 1 μg/ml CSP;

FIG. 9 shows the results of an MBEC protocol treating S. mutans biofilms with serially diluted combinations of (A) nutmeg and cranberry extracts; (B) nutmeg and neem extracts; (C) cranberry and neem extracts; and (D) nutmeg, cranberry and neem extracts in the presence of 0, 0.5, 1, and 2 μg/ml CSP;

FIG. 10 shows the results of an MBEC protocol treating S. mutans biofilms with serially diluted extracts of nutmeg, cranberry, and neem in the presence of 0, 0.5, 1, and 2 μg/ml CSP;

FIG. 11 shows the results of an MBEC protocol treating S. mutans biofilms with serially diluted combinations of nutmeg and cranberry extracts in the presence of 0, 0.5, 1, and 2 μg/ml CSP;

FIG. 12A shows the results of a bacterial selectivity test treating an S. mutans biofilm with serially diluted nutmeg extract;

FIG. 12B shows the results of a bacterial selectivity test treating a Lactobacillus casei biofilm with serially diluted nutmeg extract;

FIG. 12C shows the results of a bacterial selectivity test treating an S. oralis biofilm with serially diluted nutmeg extract;

FIG. 13A shows the results of a bacterial selectivity test treating an S. mutans biofilm with an organic solvent control;

FIG. 13B shows the results of a bacterial selectivity test treating an S. mutans biofilm with serially diluted nutmeg, cranberry, and neem extracts;

FIG. 13C shows the results of a bacterial selectivity test treating an S. oralis biofilm with serially diluted nutmeg, cranberry, and neem extracts;

FIG. 13D shows the results of a bacterial selectivity test treating an S. bovis biofilm with serially diluted nutmeg, cranberry, and neem extracts;

FIG. 13E shows the results of a bacterial selectivity test treating an S. thermophilus biofilm with serially diluted nutmeg, cranberry, and neem extracts;

FIG. 13F shows the results of a bacterial selectivity test treating an L. lactis biofilm with serially diluted nutmeg, cranberry, and neem extracts;

FIG. 13G shows the results of a bacterial selectivity test treating an L. casei biofilm with serially diluted nutmeg, cranberry, and neem extracts;

FIG. 14A shows the results of a bacterial selectivity test treating an S. mutans biofilm with a combination of serially diluted nutmeg and cranberry extracts;

FIG. 14B shows the results of a bacterial selectivity test treating an S. bovis biofilm with a combination of serially diluted nutmeg and cranberry extracts;

FIG. 14C shows the results of a bacterial selectivity test treating an L. casei biofilm with a combination of serially diluted nutmeg and cranberry extracts;

FIG. 14D shows the results of a bacterial selectivity test treating an Lactococcus lacus biofilm with a combination of serially diluted nutmeg and cranberry extracts;

FIG. 14E shows the results of a bacterial selectivity test treating an S. mitis biofilm with a combination of serially diluted nutmeg and cranberry extracts; and

FIG. 14F shows the results of a bacterial selectivity test treating an S. oralis biofilm with a combination of serially diluted nutmeg and cranberry extracts.

### DETAILED DESCRIPTION OF THE PREFERRED EMBODIMENTS

Despite many previous efforts to formulate a composition to treat or inhibit dental caries, there is still an urgent need to develop new strategies for combating the development of mature biofilms. There exists a need for an effective treatment for dental caries that selectively inhibits acidogenic bacteria such as S. mutans over beneficial oral bacteria (e.g., by facilitating proliferation of the beneficial bacteria or by inhibiting the growth rate of S. mutans while not affecting the growth rate of the beneficial bacteria) such as Streptococcus oralis. Further, there is a need for compositions directed at oral health care comprising natural ingredients such as herbal extracts, and for safer prophylactic approaches without the consequences of antibiotic treatment.

The present description includes compositions and methods for treating and/or preventing dental caries, the compositions comprising herbal extracts for selectively inhibiting caries-causing bacteria over beneficial oral bacteria (e.g., by facilitating proliferation of the beneficial bacteria or by inhibiting the growth rate of S. mutans while not affecting the growth rate of the beneficial bacteria). Further, the present description includes treating or inhibiting the formation of dental caries by providing compositions comprising natural ingredients (e.g., herbal extracts) and uses of the composition for treating or inhibiting the formation of dental caries. Still further, the present description includes treating or inhibiting the formation of dental caries by providing compositions comprising multiple herbal extracts, each herbal extract having a lower effective concentration when used in combination compared to compositions comprising the herbal extracts separately. The compositions tend to provide a new way to control S. mutans-induced dental caries, an acceptable treatment of S. mutans-induced dental caries, and acceptable control of S. mutans-induced dental caries.

In one embodiment, provided is a composition comprising an orally acceptable carrier, an extract from Myristica fragrans derived from nutmeg or mace, and an extract of Vaccinium subgenus Oxycoccus. In another embodiment, provided is a composition comprising an orally acceptable carrier, an extract from Myristica fragrans derived from nutmeg or mace, and an extract of neem. In a further embodiment, provided is a composition comprising an extract from Myristica fragrans derived from nutmeg or mace, an extract of Vaccinium subgenus Oxycoccus, and an extract of neem.

In another embodiment, provided is a composition comprising an orally acceptable carrier, an extract from Myristica fragrans derived from nutmeg or mace, and an extract of a Vaccinium subgenus other than Oxycoccus. For example, the extract of the Vaccinium subgenus can include an extract of bilberry, blueberry, and/or huckleberry. The composition can additionally include an extract of Vaccinium subgenus Oxycoccus, and/or an extract of neem.

The composition can comprise multiple herbal extracts (e.g., extracts of Myristica fragrans and Vaccinium subgenus Oxycoccus), wherein each herbal extract has a lower effective concentration when used in combination compared to compositions which include each extract separately.

In still other embodiments, any of the above compositions can include an amount of Competence Stimulating Peptide (CSP) having an amino acid sequence corresponding to SEQ ID NO:1.

The compositions can be administered to a subject to treat dental caries or to inhibit the formation of dental caries. As used throughout, the term “subject” refers to a vertebrate, preferably a mammal (e.g., a human, canine, or feline). A subject can have a disease or disorder (e.g., dental caries) or be at risk of developing a disease or disorder (e.g., dental caries).

The terms “treat”, “treating”, or “treatment” as used herein refer to the effect of an administered composition to relieve, reduce, or alleviate at least one symptom of dental caries in a subject and/or the effect of an administered composition to delay progression of dental caries in a subject. The terms also encompass the effect of an administered composition to reduce the risk of worsening dental caries.

The extracts can be present in the composition in an amount selected to inhibit the growth of Streptococcus mutans in an oral biofilm. The present description contemplates that inhibition may be due to bacteriostatic effects (i.e., stalling cell division but not killing the bacteria) and/or bactericidal effects (i.e., killing the bacteria).

Administration of effective amounts of the composition can inhibit the rate of growth of S. mutans over at least one species of non-S. mutans bacteria (e.g., S. oralis). Herein the term “rate of growth” refers to the rate of increase of cells of a given species of bacteria over time. The term “selectively inhibits” refers to a difference, or A, in the rate of growth change induced between different species of bacteria when exposed to a composition described herein. For example, in one embodiment, exposure of a mixed bacterial population (i.e., a population of bacteria comprising S. mutans and non-S. mutans bacteria) to a composition described herein causes a decrease in the rate of growth of both S mutans and non-S. mutans bacteria (e.g., S. oralis), but the decrease in the rate of growth of S. mutans is greater than the decrease in the rate of growth of non-S. mutans bacteria. Accordingly the composition selectively inhibits the rate of growth of S. mutans over non-S. mutans bacteria. In another example of selective inhibition, exposure of a mixed bacterial population to a composition described herein decreases the rate of growth of S. mutans without affecting the rate of growth of non-S. mutans bacteria. In a further example of selective inhibition contemplated by the present description, exposure of a mixed population of bacteria to a composition described herein induces non-S. mutans bacteria to proliferate (i.e., to exhibit an increased rate of growth) while decreasing or not affecting the rate of growth of S. mutans. Alternatively, the composition may increase the rate of growth of S. mutans but the increase in rate of growth of S. mutans is lower than the increase in rate of growth of non-S. mutans bacteria (e.g., S. oralis).

Myristica fragrans

The plant Myristica fragrans is a tree native to Indonesia. Two spices are derived from its fruit, nutmeg (its seed) and mace (its aril). Extracts of nutmeg have bactericidal activity against S. mutans. Without being bound by theory, macelignan, a compound found in nutmeg and mace, is an effective natural anticariogenic compound.

Vaccinium subgenus Oxycoccus

The plants of the Vaccinium genus, Oxycoccus subgenus include cranberries. Cranberries include a number of different vitamins, minerals, and polyphenols. Compounds derived from cranberry fruit have beneficial effects in people suffering from urinary tract infections. Cranberry derived compounds are thought to act by preventing bacteria from attaching to the urinary tract. Without being bound by theory, cranberry derived compounds can have similar effects on oral bacteria, e.g., inhibiting S. mutans from attaching to a tooth.

### Neem

The plant Azadirachta indica is more commonly known as the neem tree. It has been used as a major component in traditional Indian medicine (Ayurvedic medicine). Its applications have been varied; for example, it has been used to treat skin disease and as a biopesticide. Its twigs have an ability to inhibit S. mutans. Without being bound by theory, azadirachtin and nimbin, chemical compounds found in neem, can have antimicrobial properties.

### Competence Stimulating Peptide (CSP)

In some embodiments the compositions described herein include amounts of S. mutans Competence Stimulating Peptide (CSP). As noted above, S. mutans is the primary etiological agent of dental carries. S. mutans has a quorum sensing system mediated by a competence-stimulating peptide (CSP). The quorum sensing system is a signal transduction system that is encoded by the comCDE genes. The comC gene encodes a CSP precursor, which acts as an autoinducer. The precursor contains a leader sequence having a conserved double glycine motif. During secretion, the N-terminal leader peptide is cleaved off by the proteolytic activity of the transporter to generate a mature peptide that is 21 residues long (CSP-21) with the amino acid sequence: SGSLSTFFRLFNRSFTQALGK (SEQ ID NO:1). The comD gene encodes comdD, a histidine kinase that is the receptor for CSP. The comE gene encodes a response regulator.

Competitive inhibitors of CSP have been investigated in relation to a method for the treatment of caries. Specifically, compounds that competitively inhibit the binding of CSP to S. mutans histidine kinase have been studied. CSP is specific for S. mutans and functions exclusively as a regulator of many genes such as gtfB and gtfC and leads to repression of gtfB and gtfC gene expression. Glucosyltransferases (GTFs) are the products of these genes and are essential for efficient attachment of S. mutans to the surface of teeth. In the presence of their substrate (sucrose), GTFs catalyze the formation of long polymers of glucose called glucans, which are instrumental in the adherence of S. mutans to the surface of teeth. Once attachment is completed, the gtf genes are repressed and sucrose is utilized as an energy source. The catabolism of sucrose results in the production of lactic acid that damages the tooth enamel and causes caries. The concentration of CSP is not naturally produced in significant quantities during the initial attachment to the tooth but is otherwise present after the adhesion is completed to ensure that gtfB and gtfC are repressed. Since there is a strong association between the number of S. mutans in the oral cavity and the predisposition for tooth decay and endocarditis, a composition that can reduce the efficiency of S. mutans adherence should reduce or even eliminate dental caries and/or endocarditis. Moreover, since early colonizing non-pathogenic oral bacteria rely on their own gtf genes for efficient adherence but are not affected by the presence of S. mutans CSP, such bacteria will gain a competitive advantage over S. mutans if the ability of S. mutans to adhere to tooth surfaces is reduced. Thus, compositions that reduce S. mutans and the efficiency of S. mutans to adhere may be useful to inhibit dental caries.

Herein the terms “Competence Stimulating Peptide” and “CSP” encompass all functionally equivalent analogs, homologs, fragments, derivatives, salts, esters, and obvious chemical equivalents, of SEQ ID NO:1. Analogs of SEQ ID NO:1 may include, but are not limited to, an amino acid sequence containing one or more amino acid substitutions, insertions, deletions and/or mutations. Amino acid substitutions may be of a conserved or non-conserved nature. Conserved amino acid substitutions involve replacing one or more amino acids of SEQ ID NO: 1 with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

CSP may be synthesized directly from its component amino acids using methods known in the art, or may be prepared using recombinant DNA methods. Accordingly, nucleic acid molecules having a sequence that encodes CSP may be incorporated according to procedures known in the art into an appropriate expression vector that ensures expression of the peptide.

In one embodiment, CSP is present in an amount selected to selectively reduce the attachment of Streptococcus mutans to teeth. CSP can be present in an amount from about 0.05% to about 50% w/w.

### Formulations

The compositions described herein may be included within a variety of dosage forms, formulations, and/or delivery agents. The composition can be formulated as a mouthwash solution. The composition can be formulated as a dentifrice. The composition can be formulated as a varnish. The composition can be formulated as a gel. The composition can be formulated as a candy or a confectionary. The composition can be formulated as an ice cream. The composition can be formulated as a chewing gum. The composition can be formulated as a syrup. The composition can be formulated as a cream. The composition can be formulated as a dentifrice gel. The composition can be formulated as a nonabrasive dentifrice gel. The composition can be formulated as a denture wash. The composition can be formulated as a denture soak. The composition can be formulated as a denture adhesive or denture cement. The composition can be formulated as a beverage. The composition can be formulated as a soft drink or sports drink. The composition can be formulated as a functional food, natural health food, snack or ration. In one embodiment a composition is formulated for topical application in the mouth. In other embodiments, the compositions can be formulated as solid or liquid oral dosage forms including a tablet, a caplet, a capsule, a chewable tablet, a quick dissolve tablet, and effervescent tablet, a hard gelatin capsule, a soft gelatin capsule, a powder, a liquid suspension, and other types of food products. One skilled in the art would recognize there are also other viable ways for delivering the composition to a subject.

Further, these compositions can be made using conventional equipment and techniques as are known in the art. One or more orally acceptable carriers can be used when preparing dosage forms incorporating the compositions. Herein the term “orally acceptable carrier” refers to any safe and effective material for use in the oral cavity of a subject. The orally acceptable carrier can comprise water. The orally acceptable carrier can comprise one or more binders. The orally acceptable carrier can comprise one or more lubricants. The orally acceptable carrier can comprise one or more disintegrants. The orally acceptable carrier can comprise one or more suspending agents. The orally acceptable carrier can comprise one or more absorbents. The orally acceptable carrier can comprise one or more preservatives. The orally acceptable carrier can comprise one or more surfactants. The orally acceptable carrier can comprise one or more colorants. The orally acceptable carrier can comprise one or more suspending agents.

Also disclosed are methods and uses for inhibiting the growth of S. mutans in a biofilm in the oral cavity of a subject, the method comprising administering to the oral cavity of the subject an effective amount of a composition described herein. Administration of the composition can selectively inhibit the growth of S. mutans relative to at least one species of non-S. mutans bacteria (e.g., S. oralis) in the biofilm. The compositions disclosed herein can be used for the preparation of an oral formulation such as those described above for the inhibition of the formation or growth of an oral biofilm. The compositions and formulations can be used to treat a condition caused by attachment of S. mutans to teeth of a subject. In one embodiment, the condition is caused by dental plaque. In a preferred embodiment, the condition is dental caries.

The methods and compositions described herein may be used for both prophylactic and treatment purposes. For prophylactic use, an effective amount of the agents described herein can be administered to a subject prior to the manifestation of dental caries or during early onset of dental caries. Administration for prophylactic purposes can be initiated several days to years prior to the manifestation of symptoms of dental caries. For treatment purposes, an effective amount of the agents described herein can be administered after diagnosis or manifestation of one or more dental caries.

According to methods taught herein, a subject can be administered an effective amount of the described compositions. The term effective amount refers to any amount necessary to produce a desired physiologic response. In one embodiment, an effective amount inhibits the growth of oral S. mutans bacteria. In another embodiment, an effective amount inhibits the rate of growth of S. mutans bacteria over the rate of growth of beneficial oral bacteria (e.g., S. oxalis). Effective amounts and schedules for administering the compositions may be determined by empirical methods; such methods for determinations are known within the art. Generally the compositions are administered at a dosage large enough to give rise to a desired effect, but not so large as to cause substantial adverse side effects, such as unwanted cross-reactions, anaphylactic reactions, and the like. Dosage can vary with the subject age, sex, type of condition being treated, route of administration, or the presence of other drugs in a treatment regimen.

### EXAMPLES

### Example 1

### Compositions for Inhibiting Dental Caries

An embodiment of a composition for inhibiting dental caries is shown in Table 1.

TABLE 1
Example composition for inhibiting dental caries.

| | % | Approx. |
|---|---|---|
| Ingredient | Range | % w/w |
| Water, purified | q.s. | q.s. |
| Glycerol | 5-20 | 10.00 |
| Xylitol | 0-10 | 5.00 |
| Polyglycitol, maltitol or sorbitol | 0-10 | 5.00 |
| Bioadhesive polymer (e.g., sodium alginate) | 0.05-1.0 | 0.20 |
| Preservative | 0-0.2 | 0.15 |
| Sodium citrate | 0-0.3 | 0.10 |
| Sodium bicarbonate | 0-1.0 | 0.50 |
| Flavour (e.g., lemon, melon) | 0.1-0.5 | 0.20 |
| Apollonia Health active agents (e.g., CSP, | q.s. | q.s. |
| natural extracts of nutmeg, cranberry, and/or | | |
| neem) | | |
| | | 100.00% |

### Example 2

### Effect of Compositions on S. mutans Growth and Biofilm Formation

### Bacterial Strains and Growth Conditions

Strepococcus mutans UA159 strain was used in this example. S. mutans strain UA159 was grown in Todd-Hewitt broth containing 0.3% yeast extract (THYE) at pH7.0 and was subcultured routinely on THYE agar plates and incubated at 37° C. in an anaerobic chamber (5% CO₂). In liquid media, cultures were incubated in closed screw-cap tubes without agitation at 37° C. in an anaerobic chamber (5% CO₂).

### Synthesis of CSP

Competence stimulating peptide (CSP) was synthesized with the sequence of the mature 21 amino acid CSP (SGSLSTFFRLFNRSFTQALGK (SEQ ID NO:1)).

### Extraction Methods

### Product Name: Cranberry Extract 1:2

Cranberry fruits (fresh), 1.0 kg, were crushed and the juice and residue collected. Ethyl alcohol, 2.0 L of 95%, and 0.5% sodium benzoate were added, and the mixture stirred for 1 hour, then stored at 4° C. for 24 hours. The liquid was decanted and the residue squeezed and collected, then filtered using a Whatman Filter Paper 1. The liquid product was stored in the dark at room temperature. Cranberry (frozen) extract was also prepared using 70% ethyl alcohol, acetone, and water.

### Product Name: Nutmeg Seed Extract

Nutmeg Seed Powder, 1.0 kg, and 3.60 L of 95% ethyl alcohol and 0.5% sodium benzoate were added and kept for 48 hours at room temperature. The liquid was decanted, the residue squeezed and the liquid extract collected and filtered using a Whatman Filter Paper 1. The liquid product was stored in the dark at room temperature. Nutmeg extract was also prepared using 40% ethyl alcohol, acetone, and water.

### Product Name: Neem Leaf Extract 1:2:5

Neem leaf powder, 1.0 kg, and 4.6 L of 95% ethyl alcohol and 0.5% sodium benzoate was added and kept for 48 hours at room temperature. The liquid was decanted and the residue squeezed and the liquid extract collected and filtered using a Whatman Filter Paper 1. The liquid product was stored in the dark at room temperature. Neem extract was also prepared using 40% ethyl alcohol, acetone, and water.

### Minimum Biofilm Eliminating Concentration (MBEC) Protocol

Biofilm formation by S. mutans UA159 was assayed and quantified using a modified method described previously (Li et al., J. Bacteriol. 184:2699-2708).

The growth of biofilms was initiated by inoculating 300 μl of an overnight UA159 S. mutans culture (1.2×10⁷ CFU/ml) into 30 ml of THYE medium supplemented with 1% sucrose. 150 μl of the cell culture and medium mixture was transferred into the individual wells of a 96-well microtiter plate. The plate was covered and incubated for 16 to 18 hours at 37° C. in a 5% CO₂-95% air mixture. The MBEC lid was transferred onto another plate with fresh THYE medium supplemented with 1% sucrose and incubated for 24 hr. Following this incubation, the MBEC lid was removed and placed onto another 96-well plate with 180 μl of sterile PBS buffer in each well for washing. The plate was placed on a shaker for half an hour at 37° C. The lid was then removed from the wash plate and placed onto a 96-well plate containing serial dilutions of the extracts to be tested for biofilm treatment. The treatment plate and lid were then incubated with shaking for half an hour at 37° C. When determining the effects of the solvent on the activity of the nutmeg, cranberry, and neem extracts, the treatment plate and lid were shaken for one minute at 37° C. Finally, the lid was transferred from the treatment plate to another 96-well plate containing 200 μl of growth medium in each well, and incubated for 24 hours at 37° C. in a 5% CO₂-95% air mixture. After the incubation period, the wells were observed for growth. Clear wells indicated no growth; cloudy wells indicated growth.

### CSP In Vitro Assay

An in vitro assay was performed to determine whether CSP in combination with natural extracts would show enhanced inhibitory effects on S. mutans growth and biofilm formation. In cases where the effects of CSP were tested, CSP was added to the serial dilution of the extract at concentrations ranging from 0.5 μg/ml to 2 μg/ml.

### Results

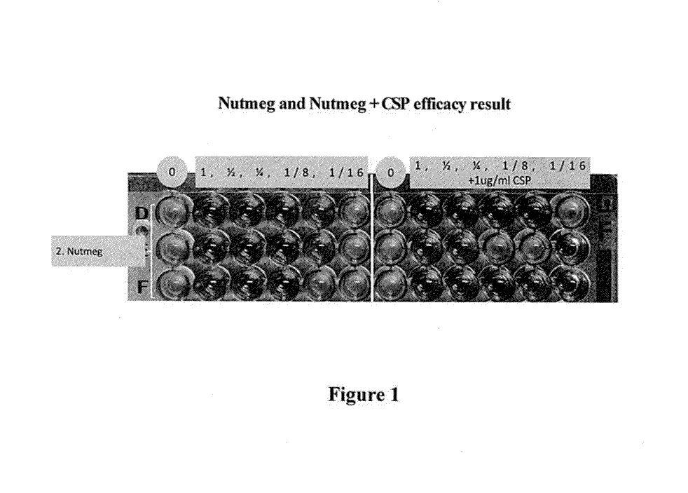

The extracts showed inhibitory effects on S. mutans growth and biofilm formation, and some extracts enhanced the inhibitory effect of CSP on S. mutans growth and biofilm formation. The results of the minimum biofilm elimination concentration assays are shown in FIGS. 1 to 5.

Nutmeg extract showed noticeable inhibition of S. mutans growth at a serial dilution of 1:8, and had a strong inhibitory effect at serial dilutions as low as 1:4 (FIG. 1; left panel). When CSP was included in the nutmeg extract (at a concentration of 1 μg/ml), inhibition of growth of S. mutans was observed at serial dilutions as low as 1:16 (FIG. 1; right panel).

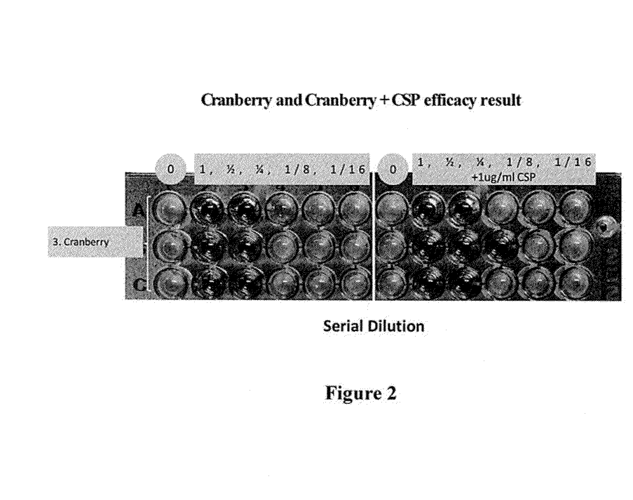

Cranberry extract inhibited growth of S. mutans at serial dilutions as low as 1:2 (FIG. 2; left panel). When CSP was included in the cranberry extract (at a concentration of 1 μg/ml), S. mutans growth was inhibited at serial dilutions as low as 1:4 (FIG. 2; right panel).

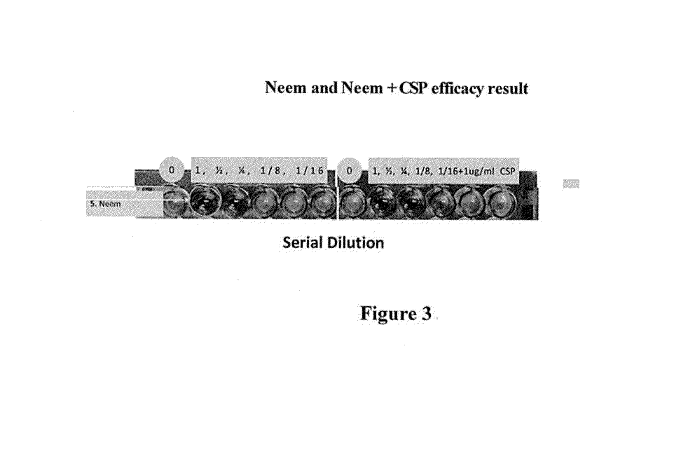

Neem extract inhibited growth of S. mutans at serial dilutions as low as 1:2 without CSP (FIG. 3; left panel) and with 1 μg/ml CSP (FIG. 3; right panel).

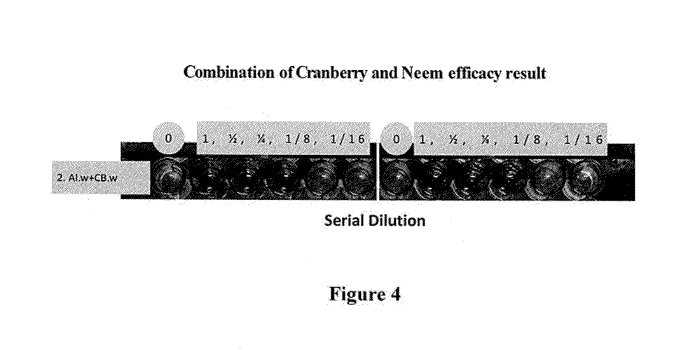

The combination of neem and cranberry extracts inhibited growth of S. mutans at serial dilutions as low as 1:4 (FIG. 4), indicating that the combined extract is twice as effective than either the neem extract (FIG. 3; left panel) or cranberry extract (FIG. 2; left panel) applied on their own.

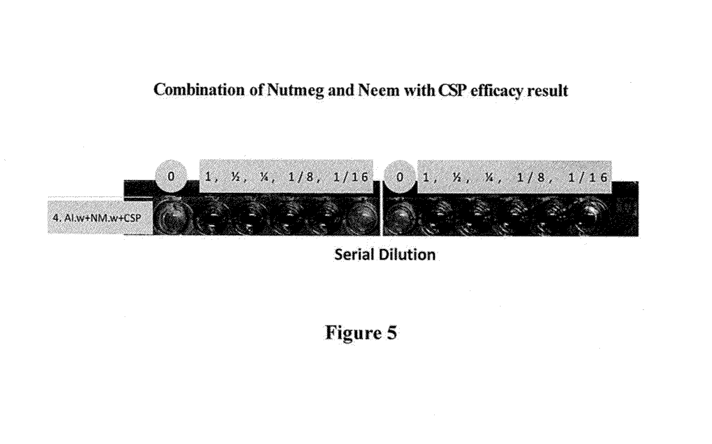

The combination of neem extract, nutmeg extract, and 1 μg/ml CSP inhibited growth of S. mutans at serial dilutions of 1:16 (FIG. 5), indicating that the combined extract is more than twice as effective as neem used alone or with CSP (FIG. 3).

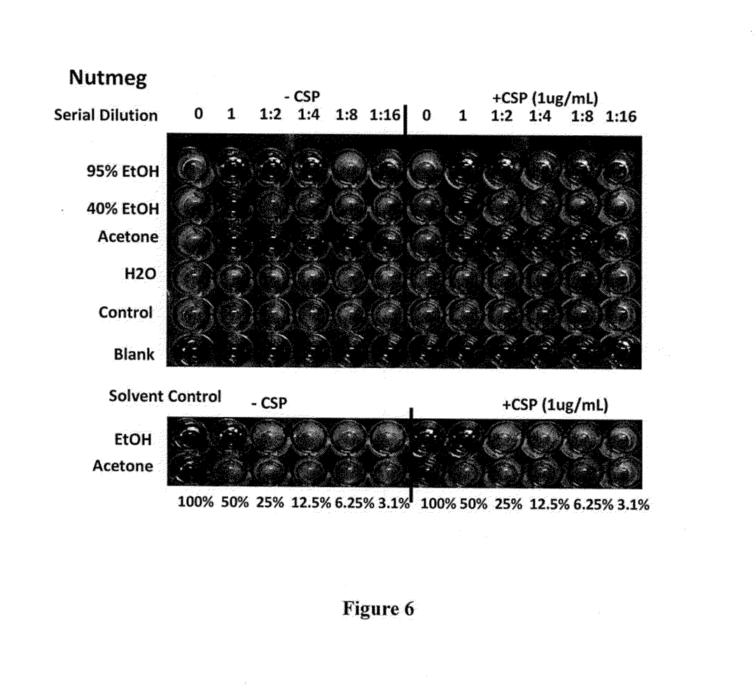

With reference to FIG. 6, nutmeg extracted using acetone as a solvent showed a stronger inhibitory effect on S. mutans than nutmeg extracted using ethanol or water as solvents. The acetone-extracted nutmeg inhibited growth of S. mutans at serial dilutions of the raw extract as low as 1:8. The absence or presence of CSP did not appear to contribute to the inhibitory effect. It is believed that the culture was not allowed sufficient time for the CSP to exhibit its activity. Nutmeg extracted with ethanol (95% or 40%) also was capable of inhibiting growth of S. mutans.

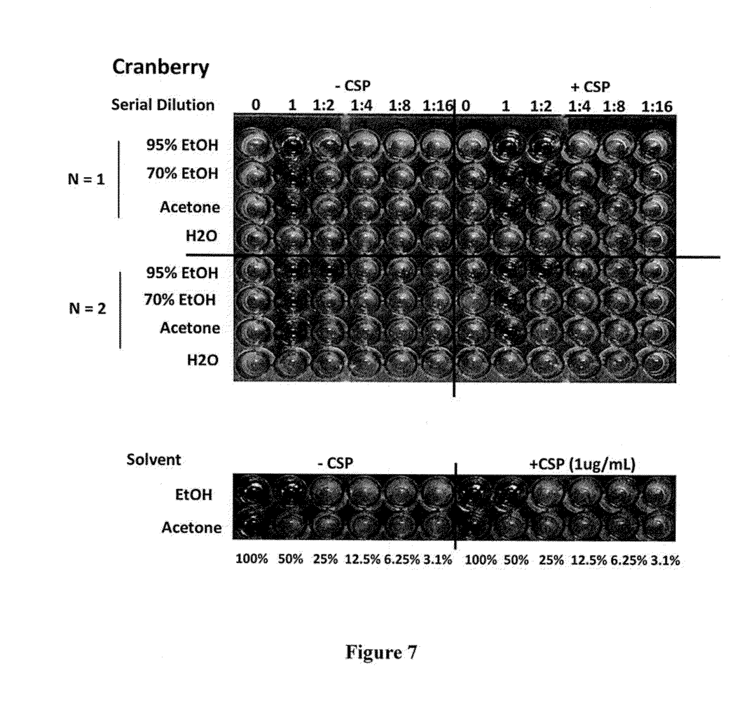

With reference to FIG. 7, cranberry extracted using 95% ethanol, 70% ethanol and acetone inhibited growth of S. mutans. Ethanol-extracted samples showed growth inhibitory activity at dilutions as low as 1:2. The organic solvents contributed at least some effect to the antibacterial activity of the cranberry extracts, as can be seen when compared to the solvent control. The absence or presence of CSP did not appear to contribute to the growth inhibition. It is believed that the culture was not allowed sufficient time for the CSP to exhibit its activity.

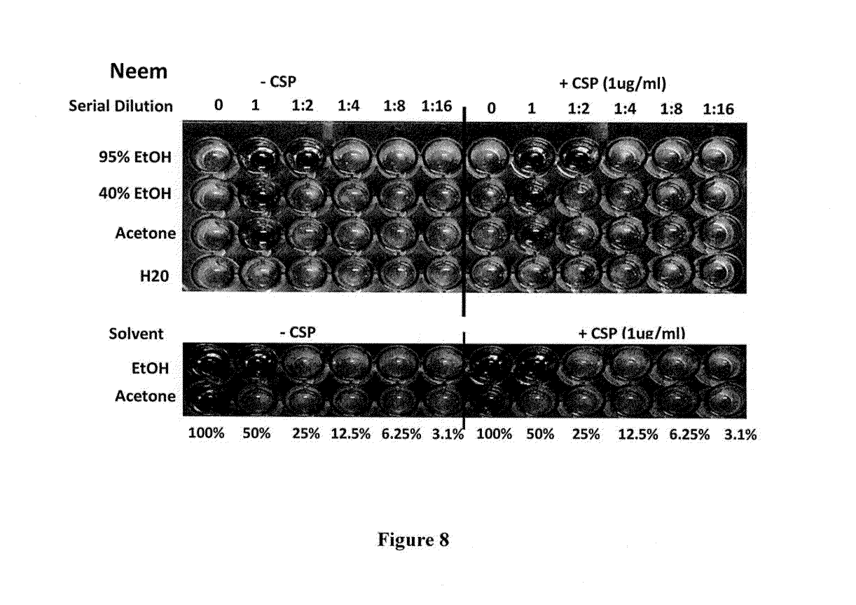

With reference to FIG. 8, neem extracted using 95% ethanol, 40% ethanol and acetone inhibited growth of S. mutans. The 95% ethanol-extracted samples showed growth inhibitory activity at dilutions as low as 1:2. The organic solvents contributed at least some effect to the antibacterial activity of the neem extracts, as can be seen when compared to the solvent control. The absence or presence of CSP did not appear to contribute to the growth inhibition. It is believed that the culture was not allowed sufficient time for the CSP to exhibit its activity.

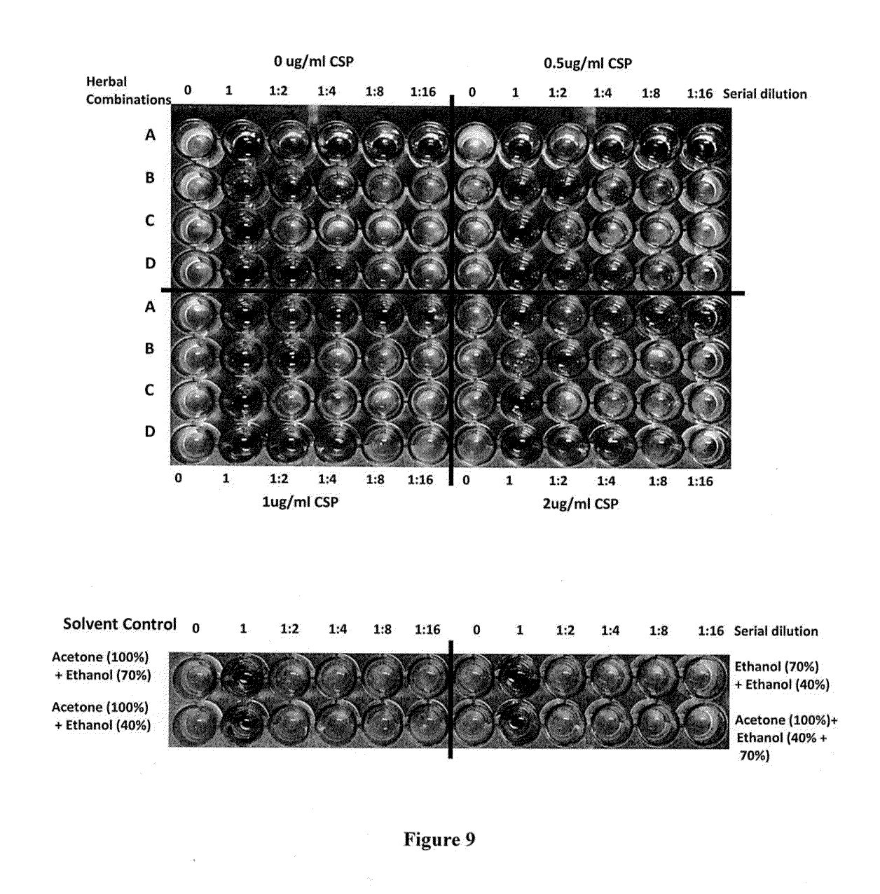

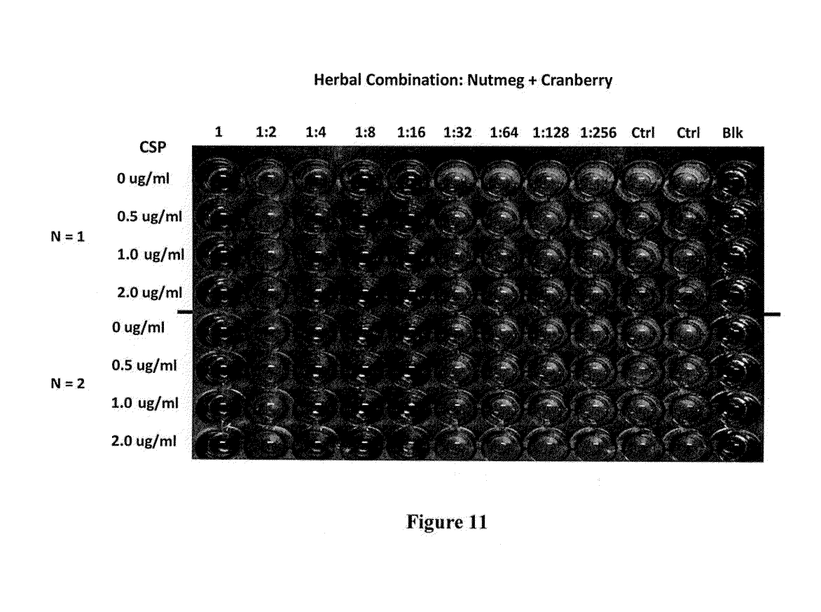

With reference to FIG. 9, the following combinations of extracts were tested: A) nutmeg and cranberry; B) nutmeg and neem; C) cranberry and neem; and D) nutmeg, cranberry and neem. The combination of nutmeg and cranberry (A) showed inhibition of S. mutans growth at dilutions of the raw extracts as low as 1:16. This combination exerted the strongest effect, although application of nutmeg and neem (B) and nutmeg, cranberry, and neem (D) combinations also gave rise to noticeable inhibition of growth. In general, the addition of CSP up to 2 μg/ml did not contribute to the inhibitory effect of combinations B and D. It is believed that the culture was not allowed sufficient time for the CSP to exhibit its activity. The effect of CSP in combination with nutmeg and cranberry extracts (A) was further investigated and the results are shown in FIG. 11.

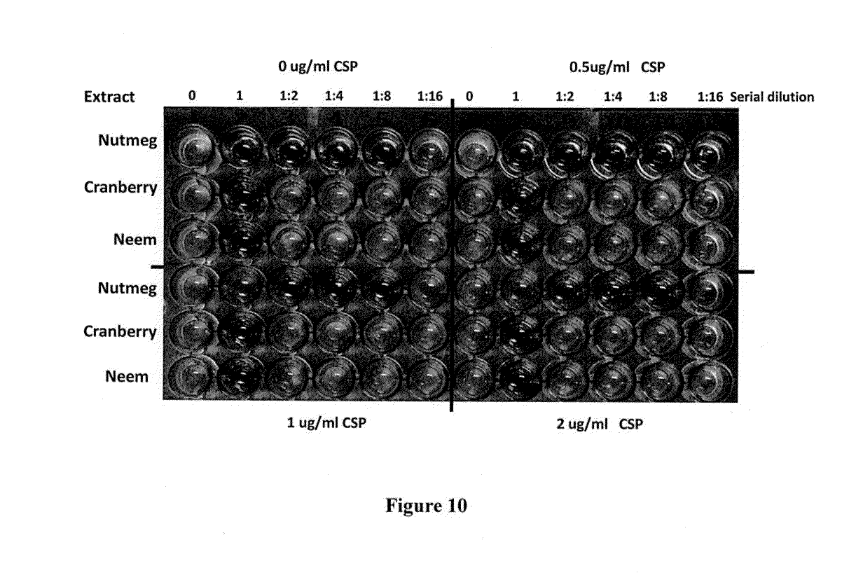

With reference to FIG. 10, the effects of the combination of extracts shown in FIG. 9 were compared to the growth inhibitory effects of each extract administered alone. Nutmeg extract alone was sufficient to inhibit growth of S. mutans at dilutions of the raw extract as low as 1:8, while raw cranberry extract was effective to inhibit growth of S. mutans only at undiluted concentrations. When nutmeg was combined with cranberry extract, the combination of extracts inhibited growth at raw extract dilutions of 1:16 (see FIG. 9A). The addition of CSP did not appear to contribute to the growth inhibitory effect. It is believed that the culture was not allowed sufficient time for the CSP to exhibit its activity. These data indicate that cranberry and nutmeg extracts interact synergistically to inhibit the growth of S. mutans. In the MBEC Examples described herein, neither nutmeg nor cranberry raw extract was observed to inhibit the growth of S. mutans when administered alone at a dilution as low as 1:16 (see e.g., left panels of FIGS. 1 and 2). However, when nutmeg and cranberry raw extracts are each present together at a dilution of 1:16, the combination of extracts inhibits growth of S. mutans. These results were unexpected and provide for a composition comprising both nutmeg and cranberry extracts which is capable of inhibiting S. mutans growth using individual extract concentrations that have no noticeable effect when administered alone. Accordingly, a composition is provided which incorporates a lower effective concentration of each of nutmeg and cranberry extracts compared to compositions which include each of these extracts separately.

With reference to FIG. 11, the combination of nutmeg and cranberry showed a growth inhibitory effect at dilutions of the raw extract as low as 1:16. The addition of CSP up to a concentration of 2 μg/ml did not appear to contribute to the growth inhibitory effect. It is believed that the culture was not allowed sufficient time for the CSP to exhibit its activity.

### Example 3

### Bacterial Selectivity Tests with THYE Agar Plates

### Bacterial Strains and Growth Conditions

Nutmeg, cranberry, and neem extracts prepared according to the extraction methods in Example 2 were tested to determine whether they had selective inhibitory effects on growth of S. mutans, S. oralis, S. bovis, S. thermophilus, Lactococcus lactis, and Lactobacillus casei. Bacteria were spread in Todd-Hewitt broth containing yeast extract (THYE) agar plates. Once the plates were dry, paper discs were overlaid on top and 30 μl of herbal extracts of different dilutions of extracts were spotted on them. Plates were incubated at 37° C. for 24 hours.

### Results

Certain extracts selectively inhibited S. mutans and L. casei growth and biofilm formation while showing less inhibition or no inhibition of S. oralis.

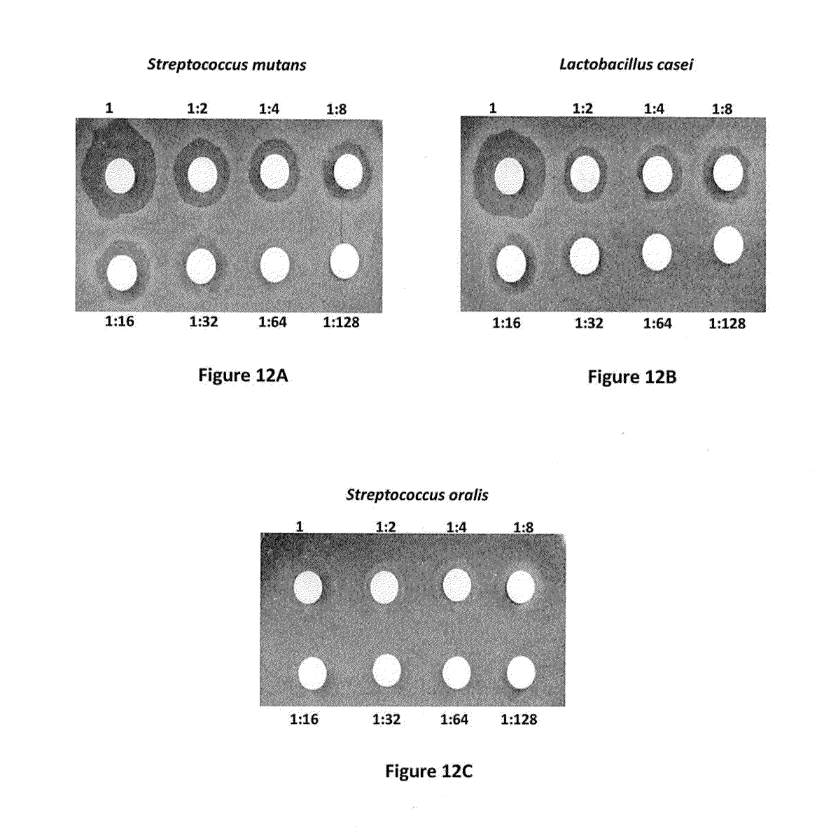

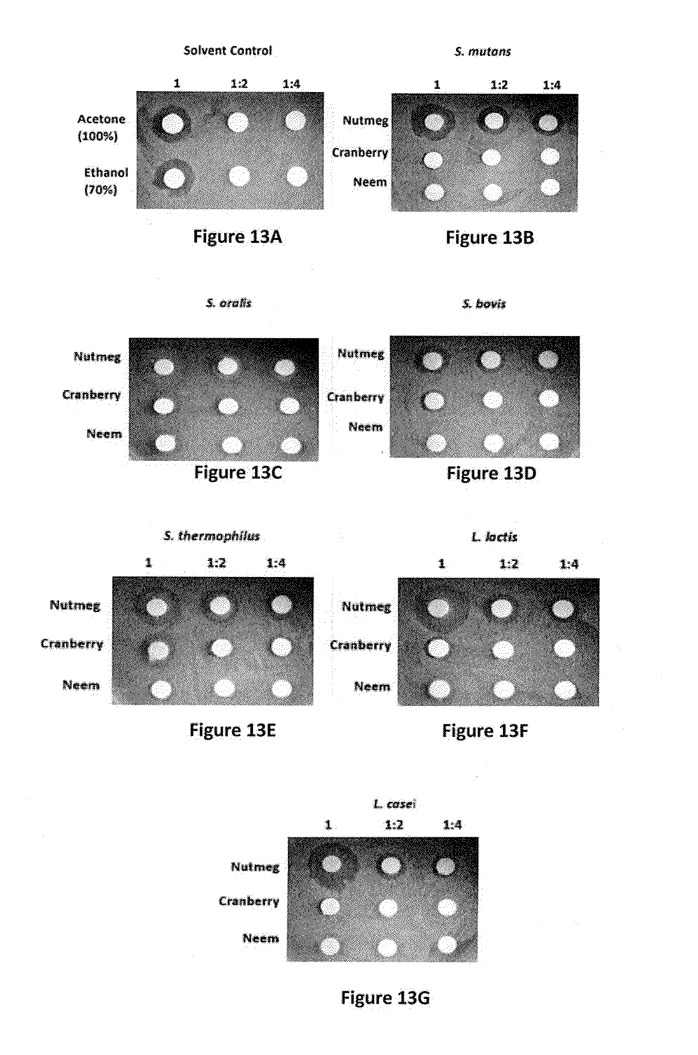

Nutmeg extract inhibited growth of S. mutans (FIGS. 12A and 13B) and L. casei (FIGS. 12B and 13G) when the concentration of the extract was at 100%, and was effective when the concentration of the raw extracts was serially diluted to as low as 1:32 (3.125%). Nutmeg extract also inhibited growth of S. bovis, S. thermophilus, and L. lactis at serial dilutions as low as 1:4 (FIGS. 13D, 13E, and 13F) relative to the solvent control (FIG. 13A). Nutmeg extract did not detectably inhibit the growth of S. oralis (FIG. 13C) at the equivalent dilutions.

Cranberry and neem extracts administered alone did not detectably inhibit the growth of any of S. mutans, S. oralis, S. bovis, S. thermophilus, L. lactis, or L. casei (FIGS. 13B-13G) relative to the solvent control (FIG. 13A).

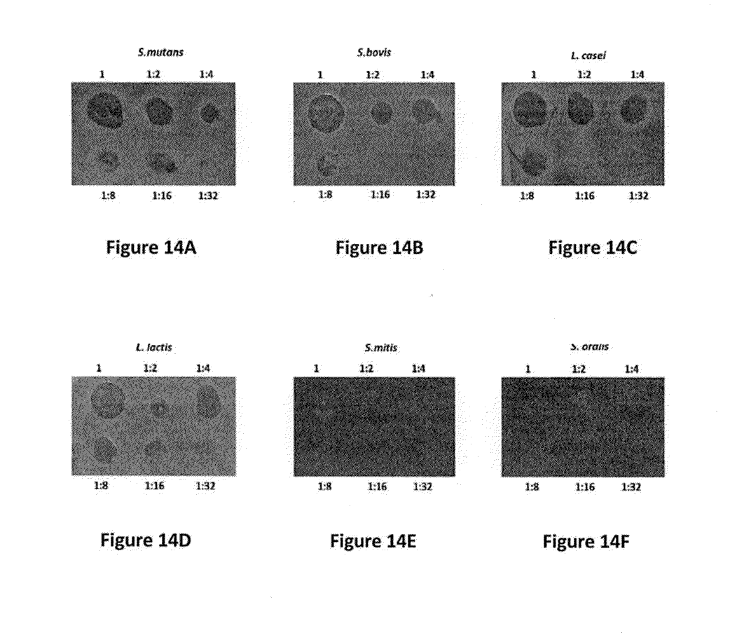

The combination of nutmeg and cranberry extracts showed inhibition of the growth of S. mutans (FIG. 14A) and L. lactis (FIG. 14D) when the concentration of the raw extract was at 100% and continued to exhibit this effect when serially diluted to 1:16. Similar growth inhibition was observed for S. bovis (FIG. 14B), and L. casei (FIG. 14C) at raw extract serial dilutions as low as 1:8. Neither S. mitis (FIG. 14E) nor S. oralis (FIG. 14F) showed detectable growth inhibition when treated with the combination of nutmeg and cranberry extracts.

### Example 4

### Bacterial Selectivity Tests with MS and MSB Plates

### Test Conditions

Herbal extracts of nutmeg, cranberry, and an equal volume mixture of nutmeg and cranberry were tested to determine whether they were selective in inhibiting the growth of S. mutans but not S. oralis. Mixed biofilms starting with equal amounts of S. mutans and S. oralis were grown overnight in ¼ THYE, 1% sucrose.

The medium was removed and the biofilms were incubated with the herbal extracts for either 1 or 30 minutes.

After the incubation periods, the herbal extract solutions were removed and THYE was added. The biofilms were then resuspended, sonicated and serial diluted in phosphate buffered saline (PBS).

Next, the samples were plated on Mitis Salivarius plates (MS) and Mitis Salivarius Bacitracin plates (MSB). Mitis Salivarius plates (MS) allow the growth of both S. mutans and S. oralis, whereas Mitis Salivarius Bacitracin plates (MSB) which contain bacitracin antibiotic and 15% sucrose are selective for S. mutans only. The plates were counted the next day.

Counts on MS plates represented a combined count of S. mutans and S. oralis. Counts on MSB plates represented a count of S. mutans only. Subtracting the count on the MSB plate from the count on the MS plate provided a count of S. oralis.

### Results

The 30 minute experiment showed that all treatments were more selective at killing S. mutans, with nutmeg showing the strongest ability to inhibit growth, followed by the mixture of nutmeg and cranberry, and then by cranberry alone.

While the subject matter described herein has been described with reference to illustrative embodiments and examples, the description is not intended to be construed in a limiting sense. Thus, various modifications of the illustrative embodiments, as well as other embodiments of the subject matter, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A composition comprising an orally acceptable carrier and:
an extract of Myristica fragrans derived from nutmeg or mace; and
an extract of Vaccinium subgenus Oxycoccus.

2. The composition of claim 1 wherein the extract of Myristica fragrans and the extract of Vaccinium are present in an amount selected to treat dental caries or inhibit the formation of dental caries by inhibiting the growth of Streptococcus mutans.

3. The composition of claim 2 wherein the extract of Myristica fragrans and the extract of Vaccinium are present in an amount selected to inhibit the growth of Streptococcus mutans in an oral biofilm.

4. The composition of claim 2 wherein the selected amount selectively inhibits the rate of growth of Streptococcus mutans over the rate of growth of at least one species of non-Streptococcus mutans bacteria.

5. The composition of claim 4 wherein the selected amount induces proliferation of the at least one species of non-Streptococcus mutans bacteria or has no effect on the rate of growth of the at least one species of non-Streptococcus mutans bacteria.

6. The composition of claim 4 wherein the selected amount decreases the rate of growth of the at least one species of non-Streptococcus mutans bacteria.

7. The composition of claim 4 wherein the Streptococcus mutans and at least one species of non-Streptococcus mutans bacteria are in a biofilm comprising a mixed bacterial population.

8. The composition of claim 4 wherein the at least one species of non-Streptococcus mutans bacteria is Streptococcus oralis.

9. The composition of claim 1 further comprising Competence Stimulating Peptide (CSP).

10. The composition of claim 9, wherein the CSP is present in an amount selected to selectively reduce the attachment of Streptococcus mutans to teeth.

11. The composition of claim 9, wherein the CSP is present in an amount ranging from about 0.05% to about 50% w/w.

12. The composition of claim 1 formulated for topical application in the mouth.

13. The composition of claim 1, wherein the composition is formulated as a denture adhesive or denture cement.

14. The composition of claim 1, wherein the composition is formulated as a candy.

15. The composition of claim 1 further comprising an extract of bilberry, blueberry, or huckleberry.

16. The composition of claim 1 further comprising an extract of neem.

17. A composition comprising an orally acceptable carrier and:
an extract of Myristica fragrans derived from nutmeg or mace; and
an extract of neem.

18. The composition of claim 17 further comprising an extract of Vaccinium subgenus Oxycoccus.

19. A method for inhibiting the growth of Streptococcus mutans in a biofilm in the oral cavity of a subject, the method comprising administering to the oral cavity of the subject a composition comprising an orally acceptable carrier and:
an extract of Myristica fragrans derived from nutmeg or mace; and
an extract of Vaccinium subgenus Oxycoccus;
wherein administering the composition selectively inhibits the rate of growth of Streptococcus mutans over the rate of growth of at least one species of non-Streptococcus mutans bacteria in the biofilm.

20. Use of a composition to inhibit the growth of Streptococcus mutans in a biofilm in the oral cavity of a subject, the composition comprising an orally acceptable carrier and:
an extract of Myristica fragrans derived from nutmeg or mace; and
an extract of Vaccinium subgenus Oxycoccus;
wherein administering the composition selectively inhibits the rate of growth of Streptococcus mutans over the rate of growth of at least one species of non-Streptococcus mutans bacteria in the biofilm.


SEQUENCE LISTING (1 sequence)

SEQ ID NO: 1 - protein, 21 aa, Streptococcus mutans
SGSLSTFFRLFNRSFTQALGK